(19) United States
(12) Patent Application Publication
(10) Pub. No.: US 2022/0218867 A1
(43) Pub. Date: Jul. 14, 2022

(54) METHODS FOR IMPROVING THE TISSUE SEALING PROPERTIES OF HYDROGELS AND THE USE THEREOF

(71) Applicant: The Regents of the University of California, Oakland, CA (US)

(72) Inventors: Amir Sheikhi, State College, PA (US); Nasim Annabi, Los Angeles, CA (US); Alireza Khademhosseini, Los Angeles, CA (US)

(73) Assignee: The Regents of the University of California, Oakland, CA (US)

(21) Appl. No.: 17/608,706

(22) PCT Filed: May 20, 2020
(86) PCT No.: PCT/US20/33775

§ 371 Date: Nov. 3, 2021

Related U.S. Application Data
(60) Provisional application No. 62/850,368, filed on May 20, 2019.

(51) Int. Cl. A61L 24/04; A61L 24/00

(52) U.S. Cl. CPC A61L 24/043; A61L 24/0042; A61L 24/0015; A61L 24/0031

(57) ABSTRACT

Naturally-derived biopolymers, such as proteins and polysaccharides are a promising platform for developing materials that readily adhere to tissues upon chemical crosslinking and provide a regenerative microenvironment. Here, we show that the sealing properties of a model biopolymer sealant, gelatin methacryloyl (GelMA), can be precisely controlled by adding a small amount of a synthetic polymer with identically reactive moieties, i.e., poly (ethylene glycol) diacrylate (PEG DA). For example, we have discovered a more than 300% improvement in tissue sealing capability of 20% (w/v) GelMA adhesive can be obtained by adding only 2-3% (v/v) PEGDA, without any significant effect on the sealant degradation time scale. These hybrid hydrogels with improved sealing properties are suitable for sealing stretchable organs, such as bladder, as well as for the anastomosis of tubular tissues/organs.

### CROSS REFERENCE TO RELATED APPLICATIONS

This application claims the benefit under 35 U.S.C. Section 119(e) of co-pending and commonly-assigned U.S. Provisional Patent Application Ser. No. 62/850,368, filed on May 20, 2019 and entitled “METHODS FOR IMPROVING THE TISSUE SEALING PROPERTIES OF HYDROGELS AND THE USE THEREOF” which application is incorporated by reference herein.

### STATEMENT OF GOVERNMENT SUPPORT

This invention was made with government support under Grant Numbers EB023052 and HL140618, awarded by the National Institutes of Health. The government has certain rights in the invention.

### TECHNICAL FIELD

The present disclosure relates to hydrogel materials useful in medical procedures such as tissue sealing.

### BACKGROUND OF THE INVENTION

Bio- and nanomaterial-assisted sutureless sealing (1-6) of tissues post-surgery provides immense advantages over conventional methods. Such materials reduce operation time and tissue damages, minimize post-operation complications (7,8), suppress inflammatory response and scar formation (9), and improve healing and regeneration (10). Tissue adhesive hydrogels are promising platforms (11) for providing a porous, moist barrier to block air or body fluids, seal leakage, inhibit bacterial infection, and permit cell infiltration to the lesion, thus facilitating wound closure and tissue regeneration. To this end, numerous adhesive materials have been explored as surgical sealants that can provide on-demand phase transition from an incision gap-filling liquid to a solid, which can seal external or internal lesions under physical and/or chemical stimuli (12-17).

Minimally invasive surgical sealants are typically prepared from synthetic polymers, natural biopolymers, or a combination of both. Synthetic polymers, such as cyanoacrylates (a clinical example: Omnex®, Ethicon, J & J) (18) and PEG (CoSeal®, Cohesion Technologies Baxter) (19) typically benefit from a precisely controllable chemical structure and robust mechanical properties. However, they do not support tissue regeneration and contribute minimally to the healing process. Naturally-derived sealants (e.g., collagen (20), gelatin (21), fibrin (22-24), albumin (25), and polysaccharides (26-29)) provide more biocompatible and biodegradable platforms, yet are affected by the heterogeneity of the material sources as well as weak mechanical and adhesive properties. Accordingly, several efforts have been devoted to developing mechanically-resilient bioadhesive hydrogel systems by using the combination of natural and synthetic polymers (30).

Typically, physical interactions are harnessed to form hydrogel tissue adhesives, including hydrogen bond formation (31,32), π-π stacking (32), ionic/electrostatic interactions (33), hydrophobic interactions (34), metal coordination (35-37), and host-guest complex formation (38.39). Common chemical modifications of macromolecules to impart stimuli-responsive properties for sealing tissues encompass functionalization with acrylate (e.g., FocalSeal from Genzyme Crop.), aldehyde (Bioglue® from Cryolife and GRF® from Cardial), phosphate, thiol, and nitrogen-containing moieties (ReSure from Ocular Therapeutix, Inc., Progel from Neomend, Inc., and Adherus from HyperBranch Medical Technology, Inc) (40). These modifications have enabled chemical binding and interlocking with connective (e.g., cartilage, bone, fat, and dense fibrous), epithelial (e.g., skin), neural, and muscular (skeletal, smooth, and cardiac) tissues (38,41). Light (visible or UV)-sensitive chemical moieties (42) in combination with adhesive moieties, such as aldehyde groups (43) and catechol (44), have been commonly used to benefit from photo-mediated on-demand radical polymerization while inducing interfacial adhesion to wet tissues.

Despite the progress so far on common hydrogel-based adhesive moieties, challenges associated with their toxicity (e.g., aldehyde-modified materials) and low mechanical properties have limited their clinical applications. For example, even though the tissue adhesion strength of mussel-inspired DOPA-functionalized GelMA (15%) increased by a factor of 4, its crosslinking efficiency and therefore the mechanical stiffness (compression modulus) decreased around eight fold (10,45). Accordingly, in the past few years, there has been a noticeable interest in developing highly adhesive mechanically resilient surgical sealants, especially for treating the injuries of stretchable organs, such as bladder, which must withstand abdominal pressure of up to 25 N during intense activities such as running and coughing (46). Another important application of sealants is the anastomosis of delicate tubes (47-54), such as blood vessels, colon, and ureter, for which sutures, staples, and adhesive tapes are all challenging to use.

Several biomaterials have recently emerged to provide non-cytotoxic wet adhesive platforms to seal injured organs. Li et al, developed stretchable adhesive patches based on pre-made dual-network polymers that were able to firmly adhere to wet organs and dissipate energy rendering the adhesive patch tough and stretchable (33). Several other efforts have also been devoted to developing adhesive patches inspired by nature (55-57). However, all of these platforms are pre-made and cannot completely fill the wound gap with irregular shapes and promote healing. GelMA has recently been used as a liquid sealant with tunable adhesion, mechanical stiffness, and degradation (10,58). Typically, increasing the concentration of prepolymer solution, increases the tensile and adhesion strength. However, high polymer concentrations lead to increased viscosity and often temperature sensitivity, significantly limiting the handling and injection of pre-gel solutions in narrow incisions. One of the unmet challenges of surgical sealants is to improve the sealing properties without compromising the native, desirable properties of pre- and post-crosslinked polymers.

Accordingly, there is a need for improved materials and methods that can be used to facilitate tissue engineering.

### SUMMARY OF THE INVENTION

The invention disclosed herein provides facile methods and materials that can be used to enhance the properties of naturally-derived tissue adhesive hydrogels without significantly changing their original desirable properties, such as biodegradation, swelling, and injectability. The technology involves adding a selected amount of a crosslinkable polymer (synthetic, semi-synthetic, and/or natural), such as polyethylene glycol diacrylate (e.g. PEGDA) to a crosslinkable naturally-derived biopolymer (such as gelatin methacryloyl, GelMA) pre-gel solution, followed by crosslinking the hybrid polymer solution on a tissue using chemical and/or photochemical reactions. The methods and materials disclosed herein can be used to modulate the material properties of a variety of medically useful hydrogels, for example to increase the sealing properties (e.g., burst pressure and wound closure strength). Consequently, the material properties of the hybrid hydrogels disclosed herein can be precisely tailored for use in a myriad of fields, including tissue sealants, hemostatic tissue adhesives, regenerative bioadhesives, and localized drug/gene/RNA delivery platforms. In addition, these hybrid bioadhesives can be used in the minimally-invasive delivery of tissue sealants through needles and/or catheters for treating internal injuries, such as bleeding.

To illustrate the applicability of the invention disclosed herein, in the sections below we detail procedures to prepare hybrid hydrogels that can be photocrosslinked (e.g. by UV or visible light) so as to form a tissue adhesive gel that can seal the defects in tissues, preventing the leakage of body fluids. These methods mix a naturally-derived biopolymer such as GelMA with a small amount of a synthetic polymer, such as PEGDA, bearing similarly reactive functional groups (e.g., Depending on the desired reaction type (e.g., photoinitiation or chemical initiation), initiators (e.g., Eosin Y, triethanolamine (TEA), and N-vinylcaprolactam (VC) for visible light crosslinking) are added to the polymer mixture. The polymer mixture can then be disposed (e.g. pipetted/injected) on tissue in situ and/or in vivo and then crosslinked (e.g., by visible light exposure for 4 min). In illustrative embodiments of the invention, the pre-gel solution forms a solid cast adhering to the tissue as a result of the crosslinking.

As discussed in detail below, embodiments of the invention include the hybrid hydrogel compositions disclosed herein as well as methods for making and using them. Briefly, embodiments of the invention include for example, compositions of matter comprising a crosslinkable biopolymer, a crosslinkable synthetic or semisynthetic polymer, polymeric monomers; and a crosslinking agent. Typically in these compositions, amounts of the crosslinkable biopolymer and amounts of the crosslinkable synthetic or semi-synthetic polymer are selected so that a hybrid hydrogel formed by crosslinking the reagents in this composition produces a hybrid biopolymer coupled to from 0.5-8% of the synthetic or semi-synthetic polymer, and the hybrid polymer hydrogel further exhibits selected material properties such as a tensile modulus of at least 150-350 kPa, a compression modulus of at least 150-350 kPa; and/or a storage modulus of at least 5-10 kPa. Related embodiments of the invention include, for example, methods of adhering a first wet tissue to a second wet tissue, these methods comprising forming a composition of matter comprising a crosslinkable biopolymer (e.g. GelMA), a crosslinkable synthetic polymer (e.g. PEGDA), and a crosslinking agent; and then disposing this composition of at a site where the composition is in contact with the first wet tissue and the second wet tissue; and then crosslinking this composition of at the site where the composition is in contact with the first wet tissue and the second wet tissue so that the crosslinked composition forms a hybrid polymer hydrogel consisting of the biopolymer covalently coupled to from 0.5-8% of the synthetic polymer; and the crosslinked composition adheres the first wet tissue to the second wet tissue.

The technology disclosed herein can be used with a wide variety biopolymer materials in order to increase the sealing properties of the resultant crosslinked hydrogels, for example by improving their cohesion. The applications of this technology span (but are not limited to) the sealing and/or regeneration of muscle, bone, cartilage, eye, lung, cardiac, and other tissues. These hybrid hydrogels can also perform as hemostatic biomaterials. Hybrid hydrogels (i.e. biopolymers coupled to a small amount of a crosslinkable synthetic polymer) can also be used as cell-friendly microenvironments for healing and regeneration applications. In this context, the invention disclosed herein enables biopolymer biomaterials to benefit from a minor chemical modification that has been discovered to impart highly desirable physical and chemical properties, for example, strength and adhesion.

The methods of hybridizing natural biopolymers with a small amount of a synthetic polymer that are disclosed herein are also useful for fabricating micro- and nano-engineered tissue adhesive hydrogels with controlled adhesion, stiffness, biodegradation, and swelling. These properties are important for example in cell culture, bioprinting, and tissue regeneration applications. One exemplary application is creating tissue adhesive bioinks that can be readily extruded through a needle, catheter, or other minimally-invasive equipment, reach different tissues, and then be crosslinked and adhered to the target site for sealing, hemostatic applications, regeneration, and/or drug/gene/protein/cell delivery. In this way, one can manufacture highly adhesive, mechanically robust, yet biodegradable hybrid hydrogels with desired micro- and/or nano-features for a broad range of applications, including localized cargo depots. The well-controlled adhesive and cohesive properties of hybrid hydrogels allow them to be used in a wide variety of medical applications such as using these tissue adhesive biomaterials for hemostatic applications, drug delivery vehicles, and injectable cell carriers.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1(A)-1(F) provide data showing the synergistic chemical and structural behavior of hybrid hydrogels. FIG. 1(A): ¹HNMR spectra of GelMA and GelMA-PEGDA before and after photocrosslinking. Upon the visible-light mediated crosslinking of GelMA and/or GelMA-PEGDA solutions, the vinyl proton peaks (highlighted in grey) shrink, indicating the reaction of C═C in GelMA. FIG. 1(B): Schematic showing the functional groups of GelMA and PEGDA. FIG. 1(C): Percentage of MA crosslinking in GelMA and GelMA-PEG-DA after 4 min of photocrosslinking. The scattering intensity I(q) versus wave vector q for hybrid hydrogels containing varying concentrations of FIG. 1(D) PEGDA and FIG. 1(E) PEG, from which FIG. 1(F) ξ and ζ are obtained, which correspond to the correlation length (average mesh size) and the length scale of density fluctuations (spatial inhomogeneities), respectively.

FIGS. 2(A)-2(I) provide data showing the mechanical, physical, and rheological properties of hybrid hydrogels. FIG. 2(A): Schematic and real setup for evaluating the compression modulus of hydrogels. FIG. 2(B): Representative compression stress/strain curves for hybrid GelMA hydrogels containing 0-5% PEGDA. FIG. 2(C): Compression moduli of hybrid hydrogels obtained from a linear fit to the stress-stain curves. Increasing the PEGDA concentration increases the compression modulus. FIG. 2(D): Schematic and real setup for evaluating tensile modulus. FIG. 2(E): Representative tensile stress/strain curves for hybrid hydrogels containing 0-5% PEGDA. FIG. 2(F): Tensile moduli obtained from a linear fit to the tensile stress-stain curves. Similar to the compression moduli, increasing the PEGDA concentration increases the tensile moduli of hybrid hydrogels. FIG. 2(G): Swelling ratios of hybrid hydrogels formed by adding various concentrations of PEGDA within 24 h, showing that increasing the PEGDA concentration decreases the swelling ratio of hybrid hydrogels. FIG. 2(H): Degradation dynamics of hybrid hydrogels in DPBS containing collagenase at 37° C. FIG. 2(I): The effect of PEGDA additive on the storage modulus of hybrid hydrogels at oscillatory shear strain ˜0.1% and angular frequency ˜10 rad/s. Statistically significant differences were identified when p-values were lower than 0.05 (*p<0.05), 0.01 (**p<0.01), 0.001 (***p<0.001), and 0.0001 (****p<0.0001).

FIGS. 3(A)-3(D) provide data showing the wound closure and burst pressure evaluation of hybrid hydrogels. FIG. 3(A): Schematic of wound closure experiments, showing the artificial wound formation in porcine skin, followed by sealing it with the visible-light-curable hydrogel. FIG. 3(B): Adhesion strength of hybrid hydrogels containing 20% GelMA and various PEGDA concentrations obtained from the wound closure experiments. The adhesion strength increased up to a PEGDA concentration ˜2-3%, followed by a decrease when the PEGDA concentration increased beyond 5%. FIG. 3(C): Schematic of burst pressure experiments, showing the perforation of a wet collagen sheet, followed by sealing it with the hybrid hydrogel. FIG. 3(D): The burst pressure of hybrid hydrogels containing 20% GelMA and various PEGDA concentrations. All the hydrogels were crosslinked via visible light exposure for 4 min. Statistically significant differences were identified when p-values were lower than 0.05 (*p<0.05), 0.01 (**p<0.01), 0.001 (***p<0.001), and 0.0001 (****p<0.0001).

FIGS. 4(A)-4E provide data showing Ex vivo sealing capability of hybrid hydrogels. FIG. 4(A): A porcine bladder is perforated, and the sealant is applied to the wound, followed by minimally invasive visible light mediated photocrosslinking (i-vi). FIG. 4(B): The bladder is connected to a flow system, and the buildup pressure by adding PBS was measured in real time. FIG. 4(C): The pressure at which the sealant fails, i.e., burst pressure, versus PEGDA concentration. A maximum resistance against PBS leakage is observed at 2% PEGDA, which is in accordance with the optimum PEGDA concentration to achieve best sealing properties, obtained from wound closure and mechanical tests. FIG. 4(D): The anastomosis capability of hybrid hydrogels was assessed by bringing two pieces of completely-torn ureter tissues together and applying the sealant, followed by light-activated crosslinking and measuring the adhesion strength using a mechanical tester (i-iv). FIG. 4(E): The adhesion strength of hybrid hydrogels anastomosing ureter versus the concentration of additive (PEGDA). The maximum adhesion strength is obtained at 2% PEGDA, which is in accordance with the standard adhesion tests (FIG. 3). Statistically significant differences were identified when p-values were lower than 0.05 (*p<0.05), 0.01 (**p<0,01), 0.001 (***p<0.001), and 0.0001 (****p<0.0001).

FIGS. 5(A)-5(C) provide data showing the mechanical and adhesion properties of hybrid GelMA-PEG (Mn=400) hydrogels. FIG. 5(A) Compression modulus, FIG. 5(B) tensile modulus, and FIG. 5(C) adhesion strength of hybrid hydrogels containing a varying PEG concentration. Increasing the PEG content to 2% does not significantly affect the mechanical and adhesion properties, showing that the presence of crosslinkable moieties, such as DA, is essential in developing superior hybrid GelMA sealants.

FIGS. 6(A)-6(C) provide data showing the properties of hybrid GelMA-PEG. FIG. 6(A): Swelling ratio of GelMA hydrogels including varying PEGDA concentrations after 24 h incubation in PBS at 37° C. FIG. 6(B): Hydrogel mass remained after 30 days of collagenase (0.5 U/mL)-mediated degradation at 37° C. FIG. 6(C): Images of hybrid hydrogels undergoing collagenase (0.5 U/mL)-mediated degradation at 37° C.

FIGS. 7(a)-7(D) provide data showing the rheological properties of hybrid sealants. FIG. 7(A) storage and FIG. 7(B) show loss moduli versus oscillatory shear strain. The strain sweep established the LVR, showing that up to at least 1% strain, the hydrogels behave linearly at an angular frequency 10 rad/s. FIG. 7(C) show storage and FIG. 7(D) loss moduli versus angular frequency. The storage modulus of hydrogels remains almost unchanged in a wide range of angular frequency (0.1-100 rad/s), a typical solid-like behavior. Increasing the PEGDA concentration increases the storage modulus, which is in accordance with the synergistic effect of PEGDA in forming a stronger network with GelMA compared to the PEG-DA-free system.

FIG. 8 provides data showing the buildup pressure versus time obtained from the burst pressure experiments conducted with hybrid hydrogels containing varying concentrations of PEGDA. The maximum (burst) pressure was obtained when the PEGDA concentration was 2%.

FIG. 9 shows a schematic summarizing aspects of the invention. The left panel shows a graph of hybrid polymer sealing performance versus PEGDA concentration. The upper middle and upper right panels show cartoon schematics of the reaction components (top middle panel) and sites for use (top right panel). The lower three panels on the right show photographs of illustrative useful applications in tissues.

### DETAILED DESCRIPTION OF THE INVENTION

in the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Naturally-derived biopolymers with tissue adhesion properties are an emerging class of tissue sealants, which have gained tremendous importance due to their biocompatibility biodegradability, bioadhesion, and cost-effectiveness. Despite their advantages, they typically lack mechanical robustness, rendering them weak tissue sealants.

As discussed below, we have developed a class of injectable or sprayable adhesives, crosslinkable composite hydrogels with improved sealing properties to promote tissue adhesion in cases such as internal and external injuries, surgical interventions, and defects (cosmetic, regenerative, and anatomical). The improved hydrogels disclosed herein can be employed in circulatory, respiratory, digestive, excretory, nervous, endocrine, immune, integumentary, skeletal, and muscular systems for surgical, pelvic, neurological, abdominal, thoracic, vascular and cardiovascular, ophthalmic, orthopedic, dermal, and cosmetic applications. Other applications encompass wound closure in open or minimally-invasive medical procedures such as the surgery of access lines (central or peripheral), catheter and drain wounds, and direct replacement for suture or staples. This composite hydrogel formulation may be used to promote tissue adhesion, tissue regeneration, and blood coagulation and fibrous formations in tissues and organs. Further applications encompass filling various defects, tissues, and/or organs, e.g., in case of corneal or stromal thinning.

The invention disclosed herein has a number of embodiments. One embodiment of the invention is a method of making a hybrid hydrogel by combining together a crosslinkable biopolymer, a crosslinkable synthetic polymer and a crosslinking agent (e.g. an agent that facilitates a photochemical reaction); and then crosslinking the biopolymer to the synthetic polymer, so that the hybrid hydrogel is formed. Biopolymers useful in aspects of the invention include FDA approved biopolymers known in the art (e.g. gelatins). Similarly, synthetic polymers useful in aspects of the invention include FDA approved synthetic polymers known in the art (e.g. polyethylene glycols).

In certain embodiments of the invention, the biopolymer comprises at least one of a gelatin, an albumin, an alginate, a chitosan, a pectin, a cellulose, any other polysaccharide, a fibrin, a collagen, or the like, and this biopolymer has a first moiety that is crosslinkable to a second moiety on another biopolymer, or on the synthetic or semi-synthetic polymer. In certain embodiments of the invention, the synthetic polymer comprises a polyethylene glycol, a polypropylene glycol, a cyanoacrylate, a poly(N-isopropylacrylamide) or the like having chemical groups that allow them to be coupled to the biopolymers (e.g. vinyl moieties). The hybrid hydrogel is typically designed to include selected amounts of synthetic polymer, amounts which significantly improve its cohesive properties without compromising other properties, such as biodegradation. As disclosed herein, there exists a non-trivial, never-reported optimum concentration of polymer additive (e.g., PEGDA) beyond which the sealing capability of hybrid hydrogels drops. In this context, in some embodiments of the invention, amounts of synthetic polymer are selected so that the hybrid polymer comprises about 0.5-8% of the hybrid hydrogel (e.g. about 1%-4%, about 2%-3%, about 2% and the like). In addition, embodiments of the invention can further comprise combining a bioactive agent such as a drug, a polypeptide, a polynucleotide or a cell with the crosslinkable biopolymer and the crosslinkable synthetic polymer.

Typically, in embodiments of the invention, the hybrid hydrogel is crosslinked in situ or in vivo such that the hybrid hydrogel forms a solid cast that adheres to wet tissues contacting the hybrid hydrogel. Optionally, amounts of synthetic polymer are selected to modulate a material property of the biopolymer, for example so that the hybrid polymer exhibits an adhesion strength to tissues that is at least two fold greater than adhesion to tissues observed with the biopolymer not crosslinked to the synthetic polymer. In certain embodiments of the invention, amounts of synthetic polymer are selected so that the hybrid polymer exhibits a compression modulus that is at least 2 fold greater than the compression modulus of the biopolymer not crosslinked to the synthetic polymer.

Embodiments of the invention include compositions of matter comprising a crosslinkable biopolymer, a crosslinkable synthetic or semisynthetic polymer, polymeric monomers (e.g. a methacrylic anhydride and/or a vinyl caprolactam), and a crosslinking agent. Typically in such compositions, amounts of the crosslinkable biopolymer, the crosslinkable synthetic polymer, the polymeric monomers and the crosslinking agent in the composition are such that so that, upon crosslinking, a hybrid hydrogel polymer is formed that comprises the biopolymer covalently crosslinked to 0.5-8% of the synthetic polymer. Typically in such compositions, following crosslinking, a hybrid polymer hydrogel is formed that exhibits a compression modulus that is at least 2-fold greater than the compression modulus exhibited by a hydrogel formed from the biopolymer not crosslinked to the synthetic polymer, in certain embodiments of the invention, upon crosslinking the composition forms a solid cast adhered to in vivo Iva tissues contacting the hybrid hydrogel.

Other embodiments of the invention include compositions of matter including a hybrid hydrogel comprising a biopolymer coupled to a synthetic polymer. In typical embodiments of the invention, the synthetic polymer comprises 0.5-8% of the hybrid hydrogel (e.g. about 1%-4%, about 2%-3%, about 2% and the like). The biopolymers can comprise polysaccharides and polypeptides and derivatives thereof having chemical groups that allow them to be coupled to synthetic polymers. In some embodiments of the invention, the biopolymer comprises at least one of an albumin, an alginate, a chitosan, a pectin, a cellulose, a fibrin, a collagen and a gelatin, said biopolymer having a first moiety/group that is couplable to a second moiety/group on the synthetic polymer. In some embodiments of the invention, the synthetic polymer comprises at least one of a polyethylene glycol, a polypropylene glycol or a cyanoacrylate having chemical groups that allow them to be coupled to biopolymers. In some embodiments of the invention, the hybrid hydrogel composition is in the form of a solid cast that is adhered to in vivo wet, tissues contacting the hybrid hydrogel. Typically, amounts of synthetic polymer in the hybrid hydrogel are controlled so that hybrid polymer exhibits an adhesion strength to in vivo wet tissues that is at least two fold greater than adhesion strength to in vivo wet tissues observed with the biopolymer not crosslinked to the synthetic polymer. In certain embodiments of the invention, the hybrid polymer exhibits a compression modulus that is at least 2-fold greater than the compression modulus exhibited by the biopolymer not crosslinked to the synthetic polymer.

Embodiments of the invention also include methods of using the compositions disclosed herein. In one illustration of this, these methods comprise adhering a composition disclosed herein to a wet tissue a lesion or site of trauma in vivo). Such methods include disposing a combination of materials disclosed herein (e.g. a crosslinkable biopolymer, a crosslinkable synthetic polymer, a crosslinking agent, a bioactive agent and the like) on the wet tissue and then crosslinking the materials in the combination so that the composition forms a solid cast that is adhered to in vivo wet tissues contacting the hybrid hydrogel.

Embodiments of the invention include, for example, methods of adhering a first tissue interface to a second tissue interface. In typical embodiments of the invention, at least one of these tissue interfaces is a wet tissue interface (e.g. a wet dynamic tissue surface present on surfaces of anatomical features found, for example, in vasculature, heart, liver, lung and the like). These methods comprise forming a composition of matter comprising a crosslinkable biopolymer, a crosslinkable synthetic polymer, and a crosslinking agent (and optionally other ingredients such as pharmaceutical excipients, polymeric monomers, or bioactive agents); disposing this composition at a site where the composition is in contact with the first tissue interface and the second tissue interface; and then crosslinking this composition of at the site where the composition is in contact with the first tissue interface and the second tissue interface such that the crosslinked composition forms a hybrid polymer hydrogel comprising the biopolymer covalently coupled to from 0.5-8% of the synthetic polymer; and the crosslinked composition of adheres the first tissue interface to the second tissue interface. In illustrative embodiments of the invention, the biopolymer comprises gelatin methacrylate (GelMA) in amounts from 10% (w/v) to 30% (w/v); and the crosslinkable synthetic polymer comprises a poly(ethylene glycol) diacrylate (PEG-DA). In certain embodiments of the invention, the composition further comprises a bioactive agent. Typically in these methods, the reagents and reaction conditions are selected so that the hybrid polymer hydrogel exhibits selected material properties such as an adhesion strength between the first tissue interface and the second tissue interface of at least 50 kPa, at least 75 kPa or at least 100 kPa. In certain embodiments of the invention, the hybrid polymer hydrogel exhibits a tensile modulus of at least 150 kPa, at least 200 kPa, at least 250 kPa, at least 300 kPA or at least 350 kPa; and/or a compression modulus of at least 150 kPa, at least 200 kPa, at least 250 kPa, at least 300 kPA or at least 350 kPa; and/or a storage modulus of at least 5 kPa or at least 10 kPa.

Embodiments of the invention include hydrogels designed to include pharmaceutically acceptable excipients. “Pharmaceutically acceptable” means that which is useful in preparing a pharmaceutical composition that is generally safe, non-toxic, and neither biologically nor otherwise undesirable and includes that which is acceptable for veterinary as well as human pharmaceutical use. For example, “pharmaceutically acceptable salts” of a compound means salts that are pharmaceutically acceptable, as defined herein, and that possess the desired pharmacological activity of the parent compound. The hydrogel compositions of the invention may contain preservatives and/or antimicrobial agents as well as pharmaceutically acceptable auxiliary substances as required to approximate physiological conditions, such as pH adjusting and buffering agents, tonicity adjusting agents, wetting agents, detergents and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's).

Using the methods and materials disclosed herein, artisans can optimize the tissue sealing properties of naturally-derived biopolymers without significantly altering their other properties, such as biodegradation and swelling. Instead of further chemical modification of crosslinkable naturally-derived biopolymers, we added a small amount of a synthetic, biocompatible (and in many cases FDA-approved) polymers, such as PEGDA, to enhance the cohesion of hybrid hydrogels. We obtained the optimum concentration of the additive polymer (PEGDA) in which the tissue sealing capability of the gel was maximized. This optimum concentration of PEGDA is typically 2-3%, which may vary for other polymers than GelMA. Further increase in the additive concentration decreases the sealing properties of the bioadhesive gels. Hybrid hydrogels with improved sealing properties find applications in a broad spectrum of industries, including but not limited to pharmaceutical companies, hygiene and personal care industries, paint industry, biomedical companies (regenerative hydrogels, drug delivery systems, peptide and protein stabilization, immunomodulating implants, etc.), and hydrogel probes (imaging, sensing, diagnostics). As an example, sealing highly-stretchable tissues is not trivial. Our engineered hybrid hydrogels are able to well seal these tissues and provide an adhesive barrier against fluid leakage. The performance of the sealants produced using our technology is several times better than the commercially available sealants. The disclosure below provides evidence that this technology will provide artisans with a newly-emerged family of highly adhesive biodegradable hydrogels for advanced biomedical applications worldwide.

The hybrid hydrogels disclosed herein provide a number of advantages over conventional materials and methods. For example, the hybrid hydrogels disclosed herein provide a noticeably higher (up to more than one order of magnitude) adhesion strength than single-component hydrogels. In addition, the hybrid hydrogels disclosed herein can be engineered to promote cell adhesion/infiltration or prevent cell adhesion; can be engineered to provide desirable in vivo degradation; can be engineered to promote tissue regeneration; can provide blood clotting action; can be used to substitute suture in the anastomosis procedure without further adhesion to the supporting medical devices, such as stents; can be applied to the lesion on demand and be crosslinked within any desirable time scale upon visible light exposure; can be easily removed from the tissue in case of an unwanted application (non-adhesive pre-gel solution); and can be easily injected and crosslinked in minimally-invasive medical procedures.

Embodiments of the technology disclosed herein provide tissue adhesive hydrogels useful for a broad range of applications, such as localized cargo/cell delivery. Embodiments of the technology disclosed herein also enable the fine tuning of sealing properties of hydrogels without significantly changing their biodegradation, swelling, and other physical properties pertinent to successful clinical translation. Embodiments of the technology disclosed herein can also enhance the tissue sealing properties of naturally-derived hydrogels beyond the commercially available sealants using a facile hybridization. Embodiments of the technology disclosed herein can also preserve the suitable properties of naturally-derived hydrogels (e.g., biodegradation) while improving the sealing properties. Further aspects and elements of the invention are described in the following sections.

### EXAMPLES

### Example 1: Aspects and Elements of the Invention

As shown below, we have developed a facile method to promote the sealing properties of photocrosslinkable hydrogel-based sealants while preserving their original properties. Benefiting from identically-crosslinkable chemical moieties, in an illustrative working embodiment of the invention, we combine GelMA, one of the most promising sealant hydrogels, with a small amount of a synthetic polymer, poly(ethylene glycol) diacrylate (PEGDA), a widely-used biocompatible polymer. We show how the addition of PEGDA has significant effects on the sealing properties of GelMA by studying the cohesive and adhesive properties of composite hydrogels.

### 1. Brief Summary of Illustrative Embodiments of the Invention

Despite the advantages of naturally-derived sealants, including biocompatibility and biodegradability, their relatively weak sealing capabilities, particularly for highly-stretchable organs, has impaired their wide-spread use. Here, we show that the sealing properties of a model biopolymer sealant, gelatin methacryloyl (GelMA), can be precisely controlled by adding a small amount of a synthetic polymer with identically reactive moieties, i.e., poly (ethylene glycol) diacrylate (PEGDA). We report on more than 300% improvement in the tissue sealing capability of 20% (w/v) GelMA adhesive by adding only 2-3% (v/v) PEGDA without any significant effect on the sealant degradation time scale. We show these hybrid hydrogels with improved sealing properties are suitable for sealing stretchable organs, such as bladder, as well as for the anastomosis of tubular tissues, e.g., ureter.

Bio- and nanomaterial-assisted sutureless sealing of tissues post-surgery provides immense advantages over conventional methods. Such materials reduce operation time and tissue damages, minimize postoperation complications, suppress inflammatory response and scar formation, and improve healing and regeneration. Despite the progress so far on common hydrogel-based adhesive moieties, challenges associated with their toxicity (e.g., aldehyde-modified materials) and low mechanical properties have limited their clinical applications. Here, we aim to develop a facile method to enhance the sealing properties of photocrosslinkable hydrogel-based sealants while preserving their original properties, such as biodegradation.

GelMA was synthesized according to conventional methods. Hybrid hydrogels were prepared by visible-light mediated crosslinking of GelMA-PEGDA solutions, and their physical, adhesive, and chemical properties were thoroughly analyzed.

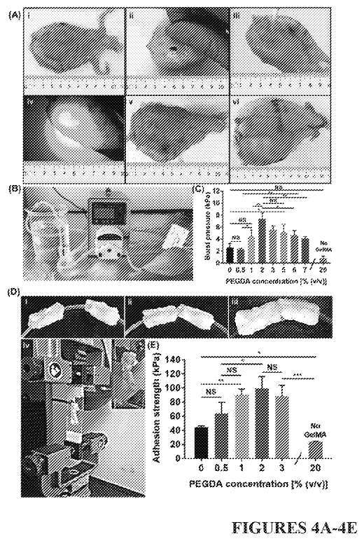

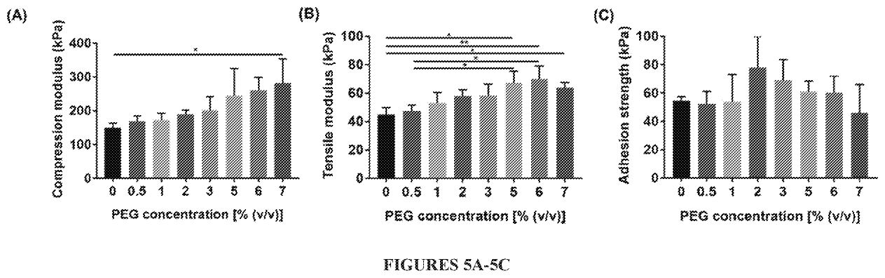

A suitable bladder sealant must resist pressure >>2 kPa. In the absence of PEGDA, the hybrid sealant could withstand ˜2 kPa, and with 2% PEGDA, the resistance against liquid pressure increased more than 300%, a very suitable property for sealing elastic, highly stretchable tissues and organs (FIG. 4A-4C). In accordance with the wound closure and standard burst pressure tests (not shown here), increasing the PEGDA concentration beyond 3% decreased the sealing capability of the hybrid hydrogels. Note that PEGDA hydrogels (20%) were not able to seal the organs. The adhesion strength of hybrid anastomotic hydrogels increased from kPa to 100 kPa by increasing the PEGDA content from 0 to 2% (FIG. 4D-4E). While PEGDA increased the cohesion of hybrid hydrogels, it decreases the tissue adhesion. PEGDA may partially consume the MA groups of GelMA, inhibiting their reaction with the tissue. Accordingly, we have unexpectedly discovered that there exists an optimum PEGDA concentration range at which the sealing capability of hybrid hydrogels is maximized, and further identified this PEGDA concentration range.

The nontrivial synergistic contribution of a photocrosslinkable synthetic polymer (PEGDA) to the sealing properties of GelMA renders it suitable for advanced sealing applications, particularly for highly stretchable tissues and organs, such as bladder and tubular architectures. Our facile approach for enhancing the sealing properties of naturally-derived hydrogels may set the stage for the next generation translational hybrid tissue sealants based on low-cost biopolymers.

### 2. Materials and Methods

### 2.1. Materials

Type-A gelatin from porcine skin (˜300 bloom), methacrylic anhydride (MA, 94%), triethanolamine (TEA, MW=149.19) vinyl caprolactam (VC, MW=139.19), Eosin Y (Mw=647.89), poly (ethylene glycol) diacrylate (PEGDA, Mn=250), and poly (ethylene glycol) (PEG. Mn=400) were provided by Sigma-Aldrich (MO, USA). Dialysis membrane with 12-14 kDa molecular weight cutoff (MWCO) was purchased from Spectrum Lab Inc (CA, USA). Milli-Q water, with an electrical resistivity of ˜18.2 MΩ cm at 25° C., was from Millipore Corporation. Polydimethylsiloxane (PDMS) base and the curing agent (SYLGARD™ 184 Elastomer Kit, Dow Corning, MI, USA) were used to construct the compression and tensile testing molds. Microscope glass slides (25 mm×75 mm×1 mm), collagenase type II and Parafilm M™ laboratory wrapping film were bought from Fisher Scientific (PA, USA). Biopsy punch was from Integra Miltex (NJ, USA). Cyanoacrylate-based adhesive was Krazy glue (Elmer's Products, NC, USA). Collagen sheet (Collagen Sausage Casing) was procured from Weston (NC, USA), and the Dulbecco's phosphate-buffered saline (DPBS, 1×) was purchased from Gibco (NY, USA).

### 2.2. Methods

### 2.2.1. Synthesis of GelMA Biopolymer

GelMA was synthesized according to our previously published articles (59,60). Briefly, porcine gelatin (10% w/v) was dissolved in DPBS at 50° C. for ˜1 h. Methacrylic anhydride (MA, 8% v/v) was then added dropwise to the solution and stirred in dark at 50° C. for 2 h. The reaction was stopped by adding an equal volume of DPBS, followed by dialysis against deionized water using 12-14 kDa cutoff dialysis tubing at 40° C. for 7 days. The final mixture was filtered (0.22 μm, VWR International, PA, USA), deep frozen at −80° C. for 24 h, lyophilized at 0.001 mbar using a freeze-drier (Free zone, 4.5 L bench top freeze drier, Labconco, MO, USA), and stored at room temperature until use. The GelMA had a high degree of methacryloyl substitution of ˜80% as confirmed by proton nuclear magnetic resonance (¹HNMR).

### 2.2.2. Preparation and Crosslinking of Hybrid Sealants

Freeze-dried GelMA was added to DPBS, containing 1.5% (w/v) TEA (co-initiator), 1% (w/v) VC (co-monomer), and 0.1 mM eosin Y (type 2 initiator), yielding a 20% (w/v) GelMA solution. The mixture was covering with aluminum foil and maintained at 80° C. for less than 30 min until the GelMA was completely dissolved. PEGDA (Mn=250) or PEG (Mn=400) was added to the mixtures at concentrations ranging from 0 to 7% (v/v), pulse-vortexed, and maintained at 37° C. for 30 min before crosslinking. To form hydrogels, the mixtures were exposed to visible light (wavelength of 450-550 nm) at an intensity of ˜100 mW/cm² for 4 min using a LS1000 Focal Seal Xenon Light Source (Genzyme Corporation, MA, USA).

### 2.2.3. Hydrogel Sample Preparation for Physical and Rheological Characterizations

To prepare the hydrogel samples for physical characterizations, 250 μL of the pre-gel mixtures were transferred to cylindrical PDMS molds (diameter ˜1 cm, height ˜3 mm) and crosslinked using visible light at an intensity of ˜100 mW/cm² for 4 min. The crosslinked samples were characterized for swelling ratio and degradation rate. The results were reported as the average of minimum 4 replicates. For the rheological characterization, hydrogels discs (diameter ˜8 mm and height ˜3 mm) were similarly prepared.

### 2.2.4. Proton Nuclear Magnetic Resonance (¹HNMR) Spectroscopy

For ¹HNMR spectroscopy, pre-gel solution and hydrogel samples were dissolved in dimethyl sulfoxide-d6 (DMSO-d6) and analyzed using Bruker ARX400 NMR. The crosslinked samples were partially solubilized in DMSO. The ¹HNMR chemical shifts were registered in parts per million (6) with respect to an internal standard, i.e., tetramethylsilane.

### 2.2.5. Small Angle X-Ray Scattering (SAXS) Measurements

SAXS measurements were performed with beamline 12-ID-B (the Advanced Photon Source. Argonne National Laboratory) at 13 keV X-rays and a 4 m sample-detector distance (q-range=0.002-0.5 Å⁻¹). The samples were loaded into 1 mm holes in aluminum plates and sandwiched between adhesive Kapton films (DuPont, USA). The samples were exposed to the beam at 25° C. for 0.1 s. The conversion of two-dimensional data to one-dimensional I(q) profiles was performed using the SAXSLee package. All further data analysis was conducted in Igor Pro.

### 2.2.6. Evaluation of Swelling Ratio

To determine the water absorption capacity of sealants, the hybrid hydrogels were lyophilized after crosslinking and their dry weight was recorded. Dry samples (n>3) were then placed in DPBS (pH=7.4) and incubated at 37° C. for 0.5, 1, 2, 4, 8, 12, and 24 h. At each time point, the samples were removed from DPBS, excess liquid was gently blotted with a tissue paper, and the wet weight was measured. The swelling ratio was calculated based on the following equation:

Swelling ratio (%)=100×(m_{w,t}−m₀)/m₀

where, m₀ and m_{w,t} are the initial dry weight of hydrogel and its weight at a given time point, respectively.

### 2.2.7. In Vitro Degradation Analysis

Freshly-crosslinked hydrogels were freeze-dried and weighed. Dry samples were placed in 4 mL of DPBS containing collagenase type II (0.5 U/mL) and incubated at 37° C. for varying periods (1, 3, 6, and 12 h, as well as 1, 2, 7, 14, 21, 30, and 60 days). The collagenase solution was replaced every 3 days to refresh the enzyme activity. At each time point, the collagenase solution was removed, samples were washed thoroughly with deionized water, lyophilized, and weighed. The shape of samples was also monitored at each time point. The degradation rate of at least three samples was determined using the following equation:

Degradation rate (%)=100×(m₀-m_{d,t})/m_{d,t}

where, m₀ and m_{d,t} are the initial dry weight of hydrogel and its dry weight at a given time point, respectively.

### 2.2.8. Rheological Characterization

Freshly-prepared hydrogels were soaked in DPBS for 24 h at room temperature to reach equilibrium swelling. The swollen hydrogels were cut using an 8 mm biopsy punch. A modular compact rheometer (MCR 302, Anton Paar, Graz, Austria) equipped with a parallel stainless-steel sandblasted plate (PP08/S, diameter ˜8 mm) was used to analyze the rheological properties of hydrogels. Oscillatory strains were imposed on the samples, and the storage modulus (G′) and the loss modulus (G′) were measured at various angular frequency and oscillatory shear strain values. All measurements were conducted at room temperature with a solvent trap installed on the rheometer to ensure minimal evaporation of the solvent. The linear viscoelastic region (LVR, i.e., the region that G′ does not significantly decrease by increasing the oscillatory strain) was determined by conducting the oscillatory strain sweeps over the strain range of 0.01-100% at an angular frequency of 10 rad/s. After defining the LVR for the hydrogels, the angular frequency dependence of viscoelastic moduli was recorded over a range of 0.1 to 100 rad/s at an oscillatory strain of 0.1%. DPBS was added onto the samples to maintain them hydrated inside the enclosed chamber during the measurements.

### 2.2.9. Mechanical Properties

The uniaxial compression and tensile tests were carried out using an Instron mechanical tester (Instron 5542, Norwood, Mass., USA). For compression tests, 250 μL of the sealant pre-gel solutions were pipetted into a cylindrical PDMS mold (diameter ˜1 cm, height ˜5 mm) and crosslinked with visible light for 4 min. The crosslinked hydrogels were then incubated in DPBS at room temperature overnight and their dimensions were measured using a digital caliper prior to the compression tests. Compression tests were performed at a strain rate ˜1 mm/min up to a strain level ˜30%. Compression moduli were calculated from the slope of linear stress-strain curves up to a strain ˜15%. For the tensile tests, 100 μL of the sealant pre-gel solution was transferred to a cuboid PDMS mold (5 mm×10 mm×1 mm), crosslinked, incubated in DPBS at room temperature for a day, followed by size measurement prior to testing. The tensile test was conducted at a strain rate ˜10 mm/min, and samples were stretched up to failure. Tensile modulus was calculated from the slope of linear stress-strain curve up to strain ˜15%. All data were reported as the mean±standard deviation of at least 5 measurements per condition.

### 2.2.10. Assessment of Scaling Properties

### In Vitro Burst Pressure Test

The burst pressures of the hybrid hydrogels were determined using the ASTM (American Society for Testing and Materials) F2392-04 standard protocol (61) with a slight modification. Briefly, collagen sheets were cut into round pieces (diameter ˜30 mm) and soaked in DPBS for 1 h at room temperature. A circular defect (diameter ˜1 mm) was created in the center of collagen sheets using a 1 mm biopsy punch. The wet collagen sheet was then placed on a piece of Parafilm, and 20 μL of a desired sealant pre-gel mixture was pipetted onto the defect and photocrosslinked by visible light for 4 min. The sealed collagen sheet was then placed into a custom-built burst pressure device, and a syringe pump was used to apply pressure by pumping air at a constant rate ˜30 mL/min. The burst pressure device was connected to a pressure sensor (Pasco Scientific, CA, USA) and the pressure was constantly recorded versus time using the SPARKvue software (version 3.2.1.3, Pasco Scientific, CA, USA). The maximum pressure at the point of rupture was recorded as the burst pressure. A minimum of 5 samples were tested for each condition, and the data were reported as the mean±standard deviation.

### Wound Closure Test

The adhesion strength of hybrid hydrogels was evaluated following the ASTM F2458-05 standard protocol (62) with some modifications. Porcine skin was purchased from a local slaughterhouse, cut into rectangular pieces (10 mm×40 mm), and soaked in DPBS for 1 h prior to the experiment. The tissue was then removed from DPBS, blotted using a tissue paper, and glued at each end on a glass slide (25 mm×75 mm) using ethyl 2-cyanoacrylate glue (Krazy glue). About 20 mm of skin remained non-glued between the two slides. The skin stripe was then cut apart from the middle of non-glued section using a razor blade to mimic a wound model. The desired sealant pre-gel mixture (50 μL) was pipetted on the incision area (1 mm×10 mm), followed by visible light-mediated crosslinking for 4 min. Finally, the two glass slides were gripped with the Instron mechanical tester and stretched at a constant strain rate ˜10 mm/min. The stress at the point of tearing was registered as the adhesion strength of sealants. A minimum of 6 replicates were tested for each hydrogel sample, and the data were reported as the mean±standard deviation.

### Ex Vivo Burst Pressure and Anastomosis

The burst pressure of hybrid hydrogels was evaluated using an ex vivo porcine bladder. Freshly dissected bladders were purchased from a local slaughterhouse or obtained from otherwise discarded animals provided by UCLA animal facility and used for the experiments within 24 h of resection to minimize necrosis-induced heterogeneity in the tissue. During the experiments, bladders were maintained moist using a wet gauze frequently soaked in DPBS. Prior to the burst pressure tests, the bladder was examined for defects by connecting it to a peristaltic pump (BT100-II, Longer Pump, NJ, USA) and pumping water at a constant rate ˜20 mL/min for 10 min to ensure there was no leakage. Thereafter, each bladder was emptied, dried with a tissue paper, and a circular incision (diameter ˜8 mm) was created on its surface by a razor blade. A desired pre-gel mixture (500 μL) was pipetted onto the incision and photocrosslinked by exposure to visible light for 4 min. Next, water was constantly pumped into the bladder using the peristaltic pump (rate ˜20 mL/min), and pressure was monitored using the wireless pressure sensor connected to the SPARKvue software. The pressure at the point of water leakage due to hydrogel rupture was recorded as the burst pressure. A minimum of 5 replicates were tested for each hydrogel, and the data were reported as the mean f standard deviation. For the anastomosis tests, porcine ureter was cut into two pieces of 4 cm long with an inner diameter of 2.5 mm. The pieces where placed together and a plastic tube (2 mm inner diameter) was inserted through the two pieces to mimic a supporting substrate during surgery. The hybrid hydrogel (30 μL) was pipetted onto the connecting area and subsequently crosslinked for 4 min using visible light. Afterward, the procedure was repeated for the opposite side of the ureter. Subsequently, the tube was removed successfully without any problem, i.e., the sealant did not adhere to it. The adhesion strength was measured using the same protocol as the wound closure experiments.

### 2.2.11. Statistical Analysis

The one-way analysis of variance (ANOVA) was conducted followed by Tukey's multiple comparisons. Statistically significant differences were noted with p-values lower than 0.05 (*p<0.05), 0.01 (**p<0.01), 0.001 (***p<0.001), and 0.0001 (****p<0.0001).

### Results and Discussion

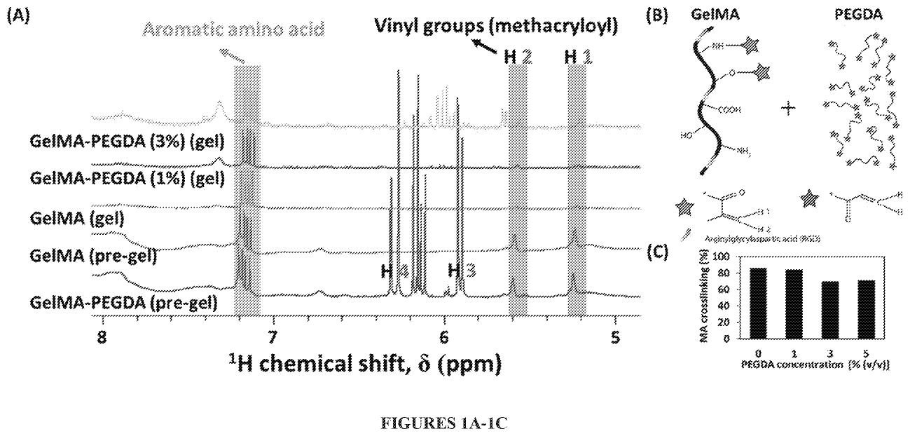

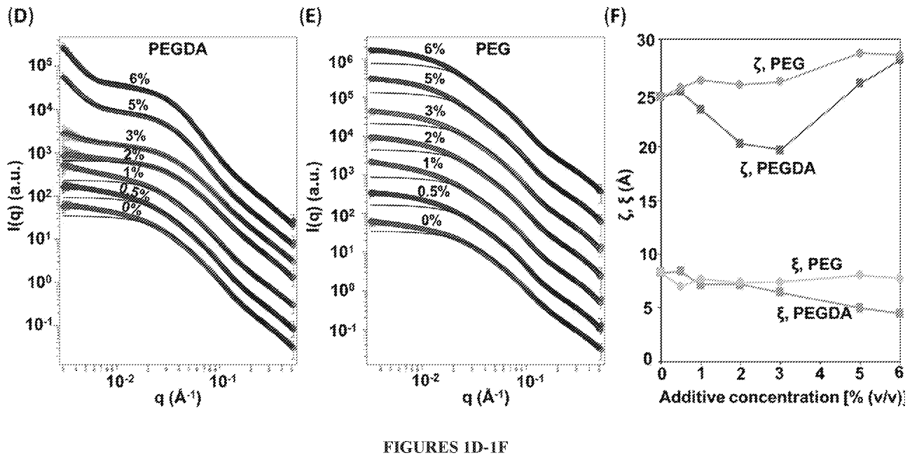

We evaluated the chemical, mechanical, and adhesive properties of the hybrid hydrogel sealants. The chemical structure of GelMA-based adhesives crosslinked under visible light was studied by conducting ¹HNMR spectroscopy on the pre-gel solutions as well as the crosslinked hydrogels partially dissolved in deuterated DMSO (FIG. 1A). The chemical structures of GelMA and PEGDA are shown in FIG. 1B. In the pre-crosslinked form of hybrid sealants, GelMA had two significant peaks between 5.25-5.75 ppm, representing the vinyl protons of MA groups. As the photocrosslinking reaction proceeded, the intensity of these two peaks normalized with the unchanged aromatic amino acid peak decreased, attesting to the MA covalent binding. After 4 min of visible light exposure, approximately 70-86% of the MA groups in the hydrogels were reacted, showing a high but not complete conversion of the reactive groups of GelMA (FIG. 1C).

To investigate the effect of PEGDA on the multiscale features of hybrid hydrogels, small angle X-ray scattering (SAXS) spectroscopy was conducted. FIGS. 1D and 1E present the scattering intensity I(q) versus wave vector q for hybrid hydrogels containing varying concentrations of PEGDA and PEG, respectively. SAXS measurements provide structural information at length scales (L) corresponding to the molecular architecture of polymer networks, with scattering intensities at larger q values corresponding to network structure at smaller length scales and vice-versa; L˜1/q. The scattering intensities from GelMA gels resembled scattering from polymer networks, with contributions both from the liquid-like concentration fluctuations with a characteristic thermal correlation length ξ that typically is observed in polymer solutions and networks and static density fluctuations arising from spatial inhomogeneities, with an inhomogeneity correlation length ζ, that is observed only in polymer networks (63-66). The overall scattering from polymer networks can thus be described as a summation of the two contributions as follows:

$$I ( q ) = \frac{{C}_{1}}{1 + {q}^{2} {ξ}^{2}} + \frac{{C}_{2}}{{( 1 + {q}^{2} {ζ}^{2} )}^{2}} \qquad ( 1 )$$

In the absence of the reactive additive (PEGDA), GelMA is partially crosslinked (˜86% of MA reacted based on the NMR spectra), forming a network of both aggregated (triple helical structure) and crosslinked (mediated by MA reaction) polymers (67). The density fluctuations with the correlation length ζ deduced from the SAXS intensity patterns were attributed to these aggregates. The scattering for the GelMA hydrogels with no additive was fit well with the model described above in Eq. (1) except for the low q region, wherein the weak power law scattering was ascribed to the large scale inhomogeneities in the hydrogels. With increasing the amount of PEGDA, the large scale inhomogeneities as well as ζ diminished. This led to a decrease in ζ with increasing additive concentration and the flattening of scattering curves at low q. These trends persisted up to 2-3% addition of PEGDA, attesting to the formation of a more homogeneous hybrid polymer network than the additive-free system. Beyond 3%, while increasing crosslinking led to a continued decrease in the mesh size ξ with increasing PEGDA, it also possibly induced significant densification of the network. This yielded significant inhomogeneities in the material and consequently the emergence of a strong, power law scattering at low q. Such phenomenon corresponds to large-scale inhomogeneities as well as an increase in (with increasing PEGDA. In comparison, when uncrosslinkable PEG was added to GelMA (FIG. 1E), ξ and ζ remained nearly constant upon the addition of PEG indicating negligible effect of PEG on the network structure. The trends in ξ and ζ are summarized in FIG. 1F, which highlight the differences of the structure of PEGDA and PEG containing GelMA hydrogels. While the addition of PEG did not affect the hydrogels in a significant manner, the addition of crosslinkable PEGDA lead initially to a homogenizing effect on the GelMA network, resulting in modest decrease in f and a pronounced reduction in (until PEGDA ˜2-3%. Further increasing PEGDA resulted in the increased inhomogeneities due to the inhomogeneous nature of crosslinked PEGDA network, resulting in an increase of (with increasing PEGDA concentrations (68).

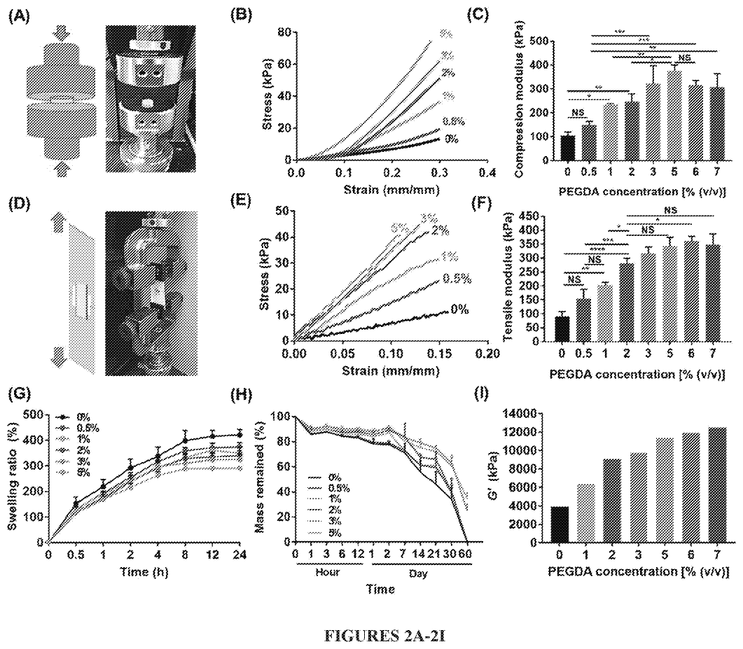

The effect of PEGDA on the crosslinking of GelMA was investigated by studying the mechanical properties of hybrid hydrogels. Compression tests were performed on the swollen GelMA hydrogel discs containing varying concentrations of PEGDA. FIG. 2A shows a schematic of compression test setup and a real sample placed on the lower (fixed) jaw of Instron ready to be compressed with a constant rate. The compression stress-strain curves of the hybrid hydrogels are shown in FIG. 2B. At a given strain, the compression stress was higher for hybrid hydrogels as compared to the GelMA hydrogel, and the stress values increased with increasing PEGDA content in the range of 0% to 5%. The corresponding compression moduli are presented in FIG. 2C, which demonstrated a four-fold enhancement in the compression modulus of hybrid gels from ˜100 kPa for pure GelMA hydrogel to ˜400 kPa, for GelMA hydrogel containing 5% PEGDA. Further increase in PEGDA concentrations (5-7%) had no significant effect on the compression moduli. A similar trend was observed for the tensile properties of hybrid hydrogels (FIG. 2D-F). The tensile (Young's) modulus of hybrid hydrogels increased with increasing PEGDA concentrations up to 5%, and then plateaued for the hydrogels with 5-7% PEGDA. The tensile modulus of hybrid hydrogels containing 2% PEGDA was more than 300% higher than pure GelMA hydrogels. The favorable contribution of PEGDA in the mechanical properties of macromolecular systems have been reported in the literature (59, 69, 70). As a control, to study the effect of PEG incorporation in the GelMA network, non-reactive PEG (Mn=400) was used. Lacking DA groups, PEG did not react with GelMA and was not covalently incorporated in the hydrogel network. FIG. 5 presents the compression modulus, tensile modulus, and adhesion strength of GelMA-PEG hybrid hydrogels. Increasing the PEG concentration up to 5% did not affect the compression modulus of hybrid hydrogels (FIG. 5-A). Similarly, up to 3% PEGDA addition had no significant effect on the tensile modulus of composite hydrogels (FIG. 5-B).

The ability to hold a large amount of water is essential for hydrogels to host cells and support their adhesion, migration, proliferation, and infiltration, all of which are pertinent to wound healing post injury. At the same time, the hydrogels should ideally degrade over time to support tissue remodeling. The swelling kinetics of dried hybrid hydrogels under the physiological condition (37° C., DPBS) over 24 h are presented in FIG. 2G. All the hydrogels almost equilibrated within the first 24 h of incubation, reaching swelling ratios between ˜300-400%. Increasing the PEGDA concentration up to 5% PEGDA monotonically decreased the swelling ratio up to 25% (FIG. 5-A). Such effects have been reported in the literature (71) and can be attributed to the increased crosslinking density upon addition of PEGDA to GelMA hydrogel networks.

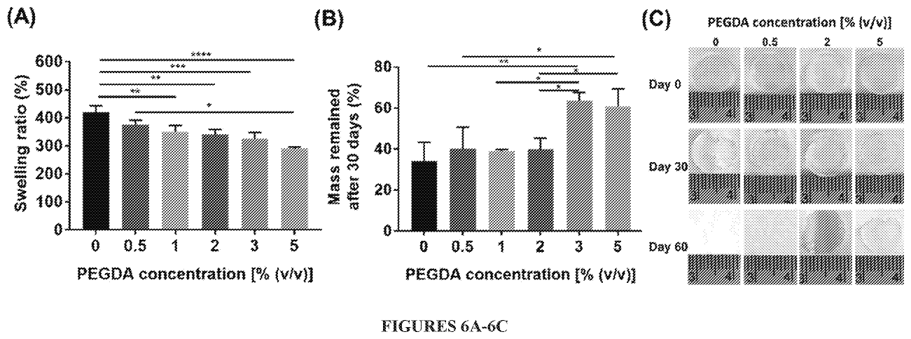

The biodegradation of hydrogels is an essential property in developing self-removable tissue sealants. FIG. 2H shows the dynamics of hydrogel degradation in the presence of collagenase (0.5 U/mL), the enzyme mainly responsible to degrade collagen in vivo. Within 30 days, the hybrid hydrogels containing up to 2% PEGDA degraded with a similar rate as PEGDA-free hydrogels (FIG. 6-B), and all hydrogels containing up to 2% PEGDA degraded completely in 60 days. At high PEGDA concentrations, e.g., 3% or 5%, more than 601% (FIG. 6-B) and 30% of the hydrogels remained within 30 and 60 days, respectively. Optical images of hybrid hydrogels undergoing degradation are presented in FIG. 6-C. Accordingly, while a low PEGDA content plays a significant role in modifying the mechanical properties of the hybrid hydrogel, it does not compromise the degradation rate governed by the natural biopolymer, GelMA.

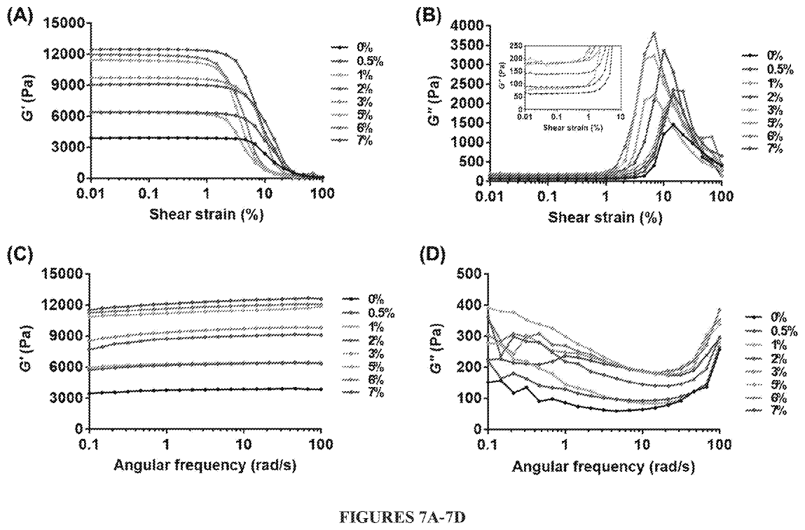

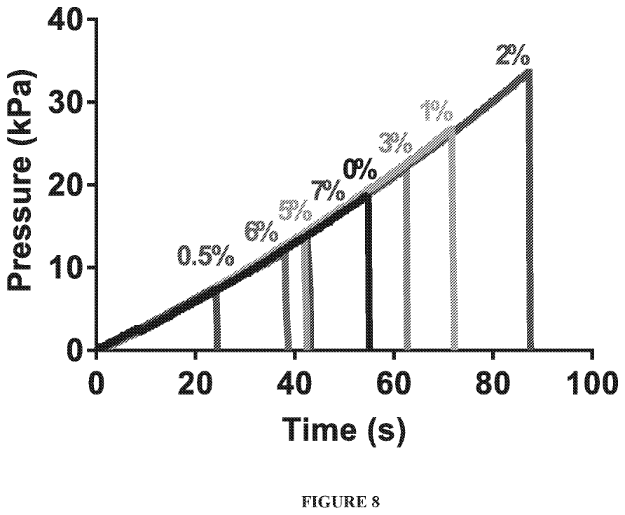

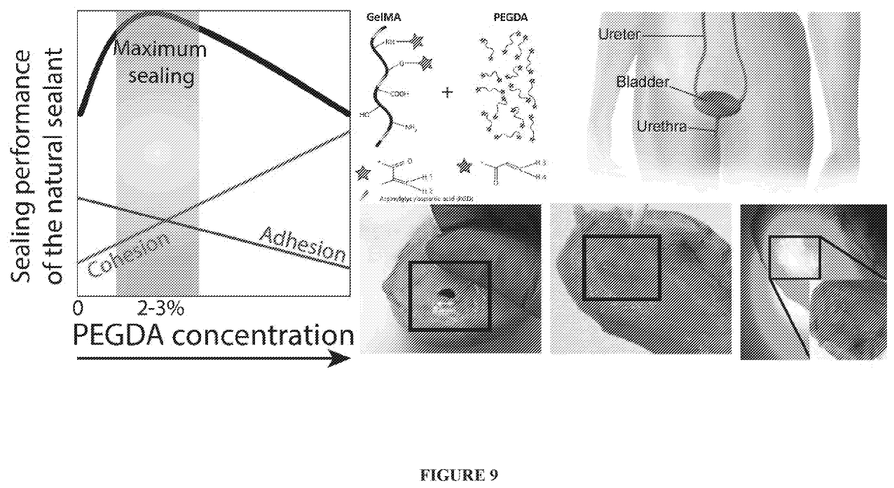

The rheological properties of hybrid hydrogels were assessed using small amplitude oscillatory rheology. The storage and loss moduli of hybrid hydrogels versus oscillatory shear strain and angular frequency are shown in FIG. 7. The storage and loss moduli of hydrogels were first measured across a wide range of oscillatory shear strain γ=0.01-100% to obtain the linear viscoelastic region (LVR) at a small angular frequency ω˜10 rad/s. Regardless of the PEGDA content, the storage modulus was not dependent on the strain when strain <1%, indicating that for all the gels, the stress correlated linearly with the strain up to at least 1% strain. In the LVR (γ=0.1%) the storage modulus of hybrid hydrogels remained nearly constant with ω ranging from 0.1 to 100 ra/s (FIG. 7-C). A comparison of the shear response of the hybrid gels was carried out by comparing the storage and loss moduli of the gels measured at ω=10 rad/s and γ=0.1% and is shown in FIG. 2I. Similar to the mechanical properties (FIG. 2A-F), the storage modulus of GelMA hydrogels increased monotonically from ˜4 kPa to ˜12 kPa with increasing PEGDA content from 0 to 7% as a result of improved crosslinking efficiency, accompanied with an increase in the loss modulus from ˜60 Pa to ˜200 Pa (FIG. 7-B). The plateau storage modulus can be associated with the characteristic mesh size ξ of an ideal network using a simplistic scaling formalism as ξ˜(G′/k_{B}T)^{-1/3}. Thus, for the hybrid hydrogels with PEGDA content ranging from 0-7%, ξ varies from ˜10 nm to ˜7 nm. These correlation length estimates agree with the estimates from SAXS measurements (FIG. 1F), especially when accounting for the inhomogeneity in the system which lead to multiple length scales as identified by SAXS in these hybrid gels. Here, k_{B} denotes the Boltzmann constant (˜1.38×10⁻²³ m² kg s⁻² K⁻¹), and T is temperature (72).

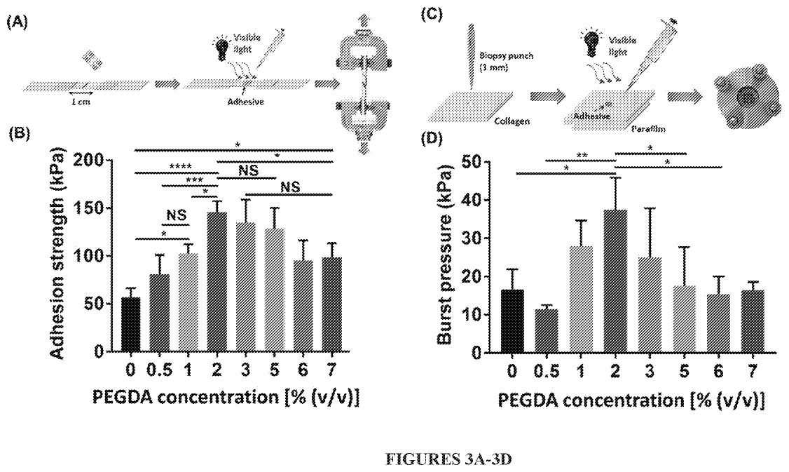

The adhesion properties of hybrid hydrogels were examined through two major standard tests, namely wound closure and burst pressure. The capability of hydrogel sealants in holding two pieces of wet porcine skin together was evaluated through the standard wound closure test (FIG. 3A). The adhesion strength of hybrid hydrogels is shown in FIG. 3B. Increasing the PEGDA concentration up to ˜2-3% increased the adhesion strength of hybrid hydrogels to the skin. Addition of PEGDA at a small concentration (e.g., 2%) increased the adhesion strength by ˜300%. Such a significant enhancement in the wound closure capability at a low additive content is non-trivial, given that PEGDA does not adhere strongly to the skin (i.e., the adhesion strength of 20% PEGDA was negligibly small). Interestingly, the adhesion strength decreased upon further increasing the PEGDA content. This indicates that the addition of a small amount of PEGDA enhanced the cohesion of the composite gels, providing an improved sealing effect. The maximum adhesion strength of hybrid hydrogels containing 2-3% (v/v) of PEGDA was ˜800% higher than the commercially available Evicel and CoSeal® sealants, and almost twice as high as that of Progel® (73).

The adhesion strength of GelMA-PEG hydrogels was not regulated by the PEG content (FIG. 5-C). These observations suggest that the synergistic effect of PEG on the GelMA sealant was contingent upon the presence of crosslinkable moieties, such as DA. At PEG concentrations >5%, a slight increase in the compression and storage modulus of hybrid GelMA-PEG hydrogels was observed, possibly as a result of increased solid content.

The capability of hybrid hydrogels in sealing air flow from a punctured collagen sheet, as a mimic of tissue, was evaluated via performing standard burst pressure experiments (FIG. 3C). Upon introducing air into the instrument, the pressure linearly increased as long as the defect is perfectly sealed, and when the sealant ruptured, the pressure immediately dropped. The maximum pressure that the sealant hydrogel can withstand is called burst pressure. FIG. 3D shows the trend of burst pressure versus PEGDA concentration. As shown in this Figure, the burst pressure data followed the wound closure results: a maximum burst pressure at ˜2-3% PEGDA, equivalent to ˜3 fold enhancement compared to the additive-free GelMA sealant was achieved, and the sealing ability of the hybrid hydrogel decreased when the PEGDA content was increased beyond 3%. The maximum burst pressure of hybrid hydrogels with 2-3% PEGDA was >600% higher than the commercially available CoSeal®, Evicel®, and Progel® sealants (73).

The ex vivo sealing properties of hybrid hydrogels were investigated in two highly challenging medical conditions, bladder and ureter ruptures. These conditions are typically results of trauma (74,75), e.g., pelvic or abdominal, which can also be a maternal and fetal life-threatening event during childbirth, especially in women with a cesarean history (76,77). The ex vivo sealing capability of hybrid hydrogels was evaluated using a flow of DPBS. An undamaged ex vivo porcine bladder was perforated, followed by placing the sealant pre-gel solution, crosslinking it using the visible light, and measuring the pressure (FIG. 4A-B). The pressure at which the sealant ruptures was measured for a variety of hybrid hydrogels containing various PEGDA concentrations (FIG. 4C). The normal pressure that the bladder requires to withstand, intra-abdominal pressure (IAP), may fall within 5-7 mmHg (0.67-0.93 kPa) (78), which may extend to 9-14 mmHg (1.20-1.87 kPa) for obese patients and >13 mmHg (1.73 kPa) for patients post-surgery (79). For patients with the head of bed (HOB) elevation ˜45°, e.g., during nursing, the IAP may reach >20±5 mmHg (2.7±0.7 kPa) (79). Intra-abdominal hypertension (IAH) is referred to bladder pressure >12 mmHg (1.6 kPa), and abdominal compartment syndrome (ACS) occurs when the IAP >20 mmHg (>2.7 kPa). Accordingly, a suitable bladder sealant must resist pressure >>2 kPa. In the absence of PEGDA, the hybrid sealant could withstand ˜2 kPa, and with 2% PEGDA, the resistance against liquid pressure increased more than 300%, a very suitable property for sealing elastic, highly stretchable tissues and organs. In accordance with the wound closure and standard burst pressure tests, increasing the PEGDA concentration beyond 2-3% decreased the sealing capability of the hybrid hydrogels. Note that PEGDA hydrogels (20%) were not able to seal the organs.

The anastomosis of ureter (ureteroureterostomy), e.g., post traumatic ureteral injuries, is one of the most common treatments of choice (80). A suitable sealant for anastomosis must be easily applied, penetrate well in the lesion gap, radially crosslink, and do not adhere to the supporting flexible plastic tube (e.g., catheter). We evaluated the performance of hybrid sealants in connecting two completely-tom pieces of ureter, brought into contact via a plastic tube support (FIG. 4D, i-iii) and assessed the adhesion strength (FIG. 4D, iv) using a mechanical tester. The adhesion strength of hybrid anastomotic hydrogels, shown in FIG. 4E, increased from ˜40 kPa to ˜100 kPa by increasing the PEGDA content from 0 to 2%. Interestingly, the maximum adhesion strength of the hybrid sealants was more than 400% higher than the commercially available Evicel sealant's adhesion strength in the anastomosis of aorta (73). While PEGDA increased the cohesion of hybrid hydrogels, it decreases the tissue adhesion. PEGDA may partially consume the MA groups of GelMA, inhibiting their reaction with the tissue. Accordingly, there exists an optimum PEGDA concentration at which the sealing capability of hybrid hydrogels is maximized.

The superior sealing properties of the hybrid hydrogels may be used in the anastomosis of other tubular tissue and organs in the circulatory, reproductive, urinary, and digestive system, such as blood vessels, Fallopian tubes, urethra, esophagus, trachea, and intestines. Relatively low cost and ease of preparation of hybrid hydrogels, compared to the emerging sealants and commercially available ones may provide a promising platform for next generation cost-effective, durable wet tissue sealants.

### CONCLUSIONS

Natural biopolymers, benefiting from biodegradability and biocompatibility, are an attractive class of macromolecules for developing surgical sealants. Despite the advances in the chemical modification of biopolymers to convert them into tissue adhesives, overcoming sub-optimal mechanical properties and tissue adhesion remain an unmet challenge. Here, we demonstrate that GelMA, an emerging class of tissue sealants, can be engineered with precisely controlled mechanical and adhesive properties using small amounts of a synthetic polymer (PEGDA) additive. Only 2-3% (v/v) of PEGDA surprisingly rendered more than 800% and 600% improvements in the adhesion strength and burst pressure of GelMA-PEGDA hybrid hydrogels, respectively, as compared to the commercially available surgical sealants, such as Evicel®. The nontrivial synergistic contribution of the synthetic polymer to the sealing properties of GelMA renders it suitable for advanced sealing applications, particularly for highly stretchable tissue and organs, such as bladder and tubular architectures. Our study suggests that the underlying mechanism for the sealing improvement at an optimum additive (PEGDA) concentration may be explained by the enhanced mechanical properties of hybrid hydrogels, and the improved hydrogel cohesion is in a tradeoff with the compromised tissue adhesion. Our facile approach for increasing the sealing properties of naturally-derived hydrogels may set the stage for the next generation hybrid tissue sealants based on low-cost biopolymers.

### REFERENCES

(1) S. J. Frost, D. Mawad, J. Hook, A. Lauto. Micro- and Nanostructured Biomaterials for Sutureless Tissue Repair, Adv. Healthc. Mater. 5 (2016) 401-414.
(2) I. C. Yasa, H. Ceylan, A. B. Tekinay, M. O. Guler, Nanomaterials as Tissue Adhesives, in: Ther. Nanomater., John Wiley & Sons, 2016: p. 173.
(3) K. Modaresifar, S. Azizian, A. Hadjizadeh, Nano/biomimetic tissue adhesives development: from research to clinical application, Polym. Rev. 56 (2016) 329-361.
(4) N. Oliva, N. Artzi, Injectable Hydrogels as Tissue Adhesives, in: Inject. Hydrogels Regen. Eng., World Scientific, 2016: pp. 239-273.
(5) R. Uric, D. Ghosh, I. Ridha, K. Rege, Inorganic Nanomaterials for Soft Tissue Repair and Regeneration, Annu. Rev. Biomed. Eng. 20 (2018) 353-374.
(6) A. P. Duarte, J. F. Coelho, J. C. Bordado, M. T. Cidade, M. H. Gil, Surgical adhesives: Systematic review of the main types and development forecast, Prog. Polym. Sci. 37 (2012) 1031-1050.
(7) S. Giordano, I. Koskivuo, E. Suominen, E. Veräjänkorva, Tissue sealants may reduce haematoma and complications in face-lifts: A meta-analysis of comparative studies, J. Plast. Reconstr. Aesthetic Surg. 70 (2017) 297-306.
(8) K. Kotzampassi, E. Eleftheriadis, Tissue sealants in endoscopic applications for anastomotic leakage during a 25-year period, Surgery. 157 (2015) 79-86.
(9) P. Fransen, Reduction of postoperative pain after lumbar microdiscectomy with DuraSeal Xact Adhesion Barrier and Sealant System, Spine J. 10 (2010) 751-761.
(10) N. Annabi, K. Yue, A. Tamayol, A. Khademhosseini, Elastic sealants for surgical applications. Eur. J. Pharm. Biopharm. 95 (2015) 27-39.
(11) V. Bhagat, M. L. Becker, Degradable Adhesives for Surgery and Tissue Engineering. Biomacromolecules. 18 (2017) 3009-3039.
(12) W. Zhu, Y. J. Chuah, D.-A. Wang, Bioadhesives for Internal Medical Applications: A Review, Acta Biomater. (2018).
(13) S. S. Kommu, R. McArthur, A. M. Emara, U. D. Reddy, C. J. Anderson, N. J. Barber, R. A. Persad, C. G. Eden. Current status of hemostatic agents and sealants in urologic surgical practice, Rev. Urol. 17 (2015) 150.
(14) A. C. Morani, J. F. Platt, A. J. Thomas, R. K. Kaza, M. M. Al-Hawary, R. H. Cohan, I. R. Francis, K. M. Elsayes, Hemostatic Agents and Tissue Sealants: Potential Mimics of Abdominal Pathologies, Am. J. Roentgenol. (2018) 1-7.
(15) I. Galanakis, N. Vasdev, N. Soomro, A review of current hemostatic agents and tissue sealants used in laparoscopic partial nephrectomy, Rev. Urol. 13 (2011) 131.
(16) F. Scognamiglio, A. Travan, I. Rustighi, P. Tarchi, S. Palmisano, E. Marsich, M. Borgogna. I. Donati, N. de Manzini, S. Paoletti, Adhesive and sealant interfaces for general surgery applications, J. Biomed. Mater. Res. Part B Appl. Biomater. 104 (2016) 626-639.
(17) H. E. Achneck, B. Sileshi, R. M. Jamiolkowski, D. M. Albala, M. L. Shapiro, J. H. Lawson, A comprehensive review of topical hemostatic agents: efficacy and recommendations for use, Ann. Surg. 251 (2010) 217-228.
(18) L. E. Jenkins, L. S. Davis, Comprehensive Review of Tissue Adhesives, Dermatologic Surg. (2018).
(19) R. Kelmansky, B. J. McAlvin, A. Nyska. J. C. Dohlman, H. H. Chiang, M. Hashimoto, D. S. Kohane, B. Mizrahi, Strong tissue glue with tunable elasticity, Acta Biomater. 53 (2017) 93-99.
(20) S. Liu, J. P. Boutrand, J. Bittoun, M. Tadie, A collagen-based sealant to prevent in vivo reformation of epidural scar adhesions in an adult rat laminectomy model, J. Neurosurg. Spine. 97 (2002) 69-74.
(21) A. Assmann, A. Vegh, M. Ghasemi-Rad, S. Bagherifard, G. Cheng, E. S. Sani, G. U. Ruiz-Esparza, I. Noshadi, A. D. Lassaletta, S. Gangadharan, A highly adhesive and naturally derived sealant, Biomaterials. 140 (2017) 115-127.
(22) F. Esposito, F. F. Angileri, P. Kruse, L. M. Cavallo, D. Solari, V. Esposito, F. Tomasello, P. Cappabianca, Fibrin sealants in dura sealing: a systematic literature review, PLoS One. 11 (2016) e0151533.
(23) S. P. Mandell, N. S. Gibran, Fibrin sealants: surgical hemostat, sealant and adhesive, Expert Opin. Biol. Ther. 14 (2014) 821-830.
(24) E. Sproul. S. Nandi, A. Brown, Fibrin biomaterials for tissue regeneration and repair, in: Pept. Proteins as Biomater. Tissue Regen. Repair, Elsevier, 2018: pp. 151-173.
(25) W. Zhu, Y. Peck, J. Iqbal, D.-A. Wang, A novel DOPA-albumin based tissue adhesive for internal medical applications, Biomaterials. 147 (2017) 99-115.
(26) S. L. Fenn, P. N. Charron, R. A. Oldinski, Anticancer Therapeutic Alginate-Based Tissue Sealants for Lung Repair, ACS Appl. Mater. Interfaces. 9 (2017) 23409-23419.
(27) B. Balakrishnan, D. Soman, U. Payanam, A. Laurent, D. Labarre, A. Jayakrishnan, A novel injectable tissue adhesive based on oxidized dextran and chitosan, Acta Biomater. 53 (2017) 343-354.
(28) M. Lu, Y. Liu, Y.-C. Huang, C.-J. Huang, W.-B. Tsai, Fabrication of photocrosslinkable glycol chitosan hydrogel as a tissue adhesive, in: Carbohydr. Polym., Elsevier. 2018: pp. 668-674.
(29) K. Azuma, M. Nishihara. H. Shimizu, Y. Itoh, O. Takashima, T. Osaki, N. Itoh, T. Imagawa, Y. Murahata, T. Tsuka, Biological adhesive based on carboxymethyl chitin derivatives and chitin nanofibers, Biomaterials. 42 (2015) 20-29.
(30) C. W. Peak, J. J. Wilker, G. Schmidt, A review on tough and sticky hydrogels, Colloid Polym. Sci. 291 (2013) 2031-2047.
(31) T. T. Hoang Thi, J. S. Lee, Y. Lee, H. B. Park, K. M. Park, K. D. Park, Supramolecular cyclodextrin supplements to improve the tissue adhesion strength of gelatin bioglues, ACS Macro Lett. 6 (2017) 83-88.
(32) L. Han, L. Yan, K. Wang, L. Fang, H. Zhang, Y. Tang, Y. Ding, L.-T. Weng, J. Xu, J. Weng, Tough, self-healable and tissue-adhesive hydrogel with tunable multifunctionality, NPG Asia Mater. 9 (2017) e372.
(33) J. Li, A. D. Celiz, J. Yang, Q. Yang, I. Wamala, W. Whyte, B. R. Seo, N. V. Vasilyev, J. J. Vlassak, Z. Suo, D. J. Mooney, Tough adhesives for diverse wet surfaces, Science (80). 357 (2017) 378-381. doi:10.1126/science.aah6362.
(34) R. Mizuta, T. Taguchi, Enhanced Scaling by Hydrophobic Modification of Alaska Pollock-Derived Gelatin-Based Surgical Sealants for the Treatment of Pulmonary Air Leaks, Macromol. Biosci. 17 (2017) 1600349.
(35) R. J. Stewart, T. C. Ransom, V. Hlady, Natural underwater adhesives. J. Polym. Sci. Part B Polym. Phys. 49 (2011) 757-771.
(36) M. Krogsgaard, V. Nue, H. Birkedal, Mussel-Inspired Materials: Self-Healing through Coordination Chemistry, Chem. Eur. J. 22 (2016) 844-857.
(37) L. Li, W. Smitthipong, H. Zeng, Mussel-inspired hydrogels for biomedical and environmental applications, Polym. Chem. 6 (2015) 353-358.
(38) C. Ghobril, M. W. Grinstaff, The chemistry and engineering of polymeric hydrogel adhesives for wound closure: a tutorial, Chem. Soc. Rev. 44 (2015) 1820-1835.
(39) A. H. Hofman, I. A. van Hees, J. Yang, M. Kamperman, Bioinspired Underwater Adhesives by Using the Supramolecular Toolbox, Adv. Mater. 30 (2018) 1704640.
(40) R. D. O'Rorke, O. Pokholenko, F. Gao, T. Cheng, A. Shah, V. Mogal, T. W. J. Steele, Addressing unmet clinical needs with UV bioadhesives, Biomacromolecules. 18 (2017) 674-682.
(41) P. J. M. Bouten, M. Zonjee, J. Bender, S. T. K. Yauw, H. van Goor, J. C. M. van Hest, R. Hoogenboom, The chemistry of tissue adhesive materials, Prog. Polym. Sci. 39 (2014) 1375-1405. doi:https://doi.org/10.1016/j.progpolymsci.2014.02.001.
(42) A. Vitale, G. Trusiano, R. Bongiovanni, UV-Curing of Adhesives: A Critical Review, Prog. Adhes. Adhes. 3 (2018) 101-154.
(43) N. Artzi, T. Shazly, A. B. Baker, A. Bon, E. R. Edelman, Aldehyde-amine chemistry enables modulated biosealants with tissue-specific adhesion, Adv. Mater. 21 (2009) 3399-3403.
(44) J. H. Ryu, S. Hong, H. Lee, Bio-inspired adhesive catechol-conjugated chitosan for biomedical applications: A mini review. Acta Biomater. 27 (2015) 101-115. doi:https://doi.org/10.1016/j.actbio.2015.08.043.
(45) H. Cheng, K. Yue, M. Kazemzadeh-Narbat, Y. Liu, A. Khalilpour, B. Li, Y. S. Zhang, N. Annabi, A. Khademhosseini, Mussel-inspired multifunctional hydrogel coating for prevention of infections and enhanced osteogenesis, ACS Appl. Mater. Interfaces. 9 (2017) 11428-11439.
(46) J. M. Shaw, N. M. Hamad, T. J. Coleman, M. J. Egger, Y. Hsu, R. Hitchcock, I. E. Nygaard, Intra-abdominal pressures during activity in women using an intra-vaginal pressure transducer, J. Sports Sci. 32 (2014) 1176-1185.
(47) A. Bracey. A. Shander, S. Aronson, B. A. Boucher, D. Calcaterra, M. W. A. Chu, R. Culbertson. K. Jabr, H. Kehlet, O. Lattouf, The use of topical hemostatic agents in cardiothoracic surgery, Ann. Thorac. Surg. 104 (2017) 353-360.
(48) V. D. Plat, B. T. Bootsma, N. van der Wielen, J. Straatman, L. J. Schoonmade, D. L. van der Peet, F. Daams, The role of tissue adhesives in esophageal surgery, a systematic review of literature, int. J. Surg. 40 (2017) 163-168.
(49) W.-G. Ma, B. A. Ziganshin, C.-F. Guo, M. A. Zafar, R S. Sieller, M. Tranquilli, J. A. Elefteriades, Does BioGlue contribute to anastomotic pseudoaneurysm after thoracic aortic surgery?, J. Thorac. Dis. 9 (2017) 2491.
(50) G. M. Bot, K. G. Bot, J. Ogumranti, J. A. Onah, A. Z. Sulc, I. Hassan, E. D. Dung, The use of cyanoacrylate in surgical anastomosis: an alternative to microsurgery, J. Surg. Tech. Case Rep. 2 (2010) 44-48.
(51) S. R. Gundry. K. Black, H. Izutani. Sutureless coronary artery bypass with biologic glued anastomoses: preliminary in vivo and in vitro results, J. Thorac. Cardiovasc. Surg. 120 (2000) 473477.
(52) H. Fonouni, A. Kashfi, A. Majlesara, O. Stahlheber, L. Konstantinidis, T. W. Kraus, A. Mehrabi, H. Oweira. Analysis of the hemostatic potential of modern topical sealants on arterial and venous anastomoses: an experimental porcine study, J. Mater. Sci. Mater. Med. 28 (2017) 134.
(53) C. D. Liu, G. J. Glantz, E. H. Livingston, Fibrin glue as a sealant for high-risk anastomosis in surgery for morbid obesity, Obes. Surg. 13 (2003) 4548.
(54) K. A. Vakalopoulos, F. Daams, Z. Wu, L. Timmermans, J. J. Jeekel, G.-J. Kleinrensink, A. van der Ham, J. F. Lange, Tissue adhesives in gastrointestinal anastomosis: a systematic review, J. Surg. Res. 180 (2013) 290-300.
(55) A. Mahdavi, L. Ferreira, C. Sundback, J. W. Nichol, E. P. Chan, D. J. D. Carter, C. J. Bettinger, S. Patanavanich, L. Chignozha, E. Ben-Joseph, A biodegradable and biocompatible gecko-inspired tissue adhesive, Proc. Natl. Acad. Sci. 105 (2008) 2307-2312.
(56) P. Rao, T. L. Sun, L. Chen, R. Takahashi. G. Shinohara. H. Guo, D. R. King, T. Kurokawa, J. P. Gong, Tough Hydrogels with Fast, Strong, and Reversible Underwater Adhesion Based on a Multiscale Design, Adv. Mater. 30 (2018) 1801884.
(57) H. Yi, S. H. Lee, M. Seong, M. K. Kwak, H. E. Jeong, Bioinspired reversible hydrogel adhesives for wet and underwater surfaces. J. Mater. Chem. B. 6 (2018) 8064-8070.
(58) E. S. Sani, A. Kheirkhah, D. Rana, Z. Sun, W. Foulsham, A. Sheikhi, A. Khademhosseini, R. Dana, N. Annabi, Sutureless repair of comeal injuries using naturally derived bioadhesive hydrogels, Sci. Adv. 5 (2019) caav1281.
(59) J. W. Nichol, S. T. Koshy, H. Bae, C. M. Hwang, S. Yamanlar, A. Khademhosseini, Cell-laden microengineered gelatin methacrylate hydrogels, Biomaterials. 31 (2010) 5536-5544.
(60) K. Yue, X. Li, K. Schrobback, A. Sheikhi, N. Annabi, J. Leijten, W. Zhang, Y. S. Zhang, D. W. Hutmacher, T. J. Klein, A. Khademhosseini, Structural analysis of photocrosslinkable methacryloyl-modified protein derivatives, Biomaterials. 139 (2017) 163-171. doi:10.1016/j.biomaterials.2017.04.050.
(61) ASTM F2392-04(2015), Standard Test Method for Burst Strength of Surgical Sealants, ASTM International, West Conshohocken, Pa., www.astm.org, 2015.
(62) ASTM F2458-05(2015), Standard Test Method for Wound Closure Strength of Tissue Adhesives and Sealants, ASTM International, West Conshohocken, Pa., www.astm.org, 2015.
(63) R. Nagarajan, B. Kalpakci, P. Dubin, Microdomains in Polymer solutions, Plenum, New York. (1985).
(64) T. Cosgrove, S. J. White, A. Zarbakhsh, R. K. Heenan, A. M. Howe, Small-angle scattering studies of sodium dodecyl sulfate interactions with gelatin. 1. Langmuir. 11 (1995) 744-749.
(65) S. Seiffert, Scattering perspectives on nanostructural inhomogeneity in polymer network gels, Prog. Polym. Sci. 66 (2017) 1-21.
(66) J. Sharma. V. K. Aswal, P. S. Goyal, H. B. Bohidar, Small-angle neutron scattering studies of chemically cross-linked gelatin solutions and gels, Macromolecules. 34 (2001) 5215-5220.
(67) A. I. Van Den Bulcke, B. Bogdanov, N. De Rooze, E. H. Schacht, M. Comelissen, H. Berghmans, Structural and rheological properties of methacrylamide modified gelatin hydrogels, Biomacromolecules. 1 (2000) 31-38.
(68) P. Malo de Molina, S. Lad, M. E. Helgeson, Heterogeneity and its Influence on the Properties of Difunctional Poly (ethylene glycol) Hydrogels: Structure and Mechanics, Macromolecules. 48 (2015) 5402-5411.
(69) K. R. Mamaghani, S. M. Naghib, A. Zahedi, M. Rahmanian, M. Mozafari, GelMa/PEGDA containing graphene oxide as an IPN hydrogel with superior mechanical performance, Mater. Today Proc. 5 (2018) 15790-15799.
(70) 1. Noshadi, B. W. Walker, R. Portillo-Lara, E. S. Sani, N. Gomes, M. R. Aziziyan. N. Annabi, Engineering biodegradable and biocompatible bio-ionic liquid conjugated hydrogels with tunable conductivity and mechanical properties, Sci. Rep. 7 (2017) 4345.
(71) C. B. Hutson, J. W. Nichol. H. Aubin, H. Bae. S. Yamanlar, S. Al-Haque, S. T. Koshy, A. Khademhosseini, Synthesis and Characterization of Tunable Poly(Ethylene Glycol): Gelatin Methacrylate Composite Hydrogels, Tissue Eng. Part A. 17 (2011) 1713-1723. doi:10.1089/ten.tea.2010.0666.
(72) P.-G. De Gennes, P.-G. Gennes, Scaling concepts in polymer physics, Cornell university press, 1979.
(73) N. Annabi, Y.-N. Zhang, A. Assmann, E. S. Sani, G. Cheng, A. D. Lassaletta, A. Vegh, B. Dehghani, G. U. Ruiz-Esparza, X. Wang, Engineering a highly elastic human protein-based sealant for surgical applications, Sci. Transl. Med. 9 (2017) eaai7466.
(74) L. V Simon, B. Burns, Bladder. Rupture, StatPearls Publ. (2017). https://www.ncbi.nlm.nih.gov/books/NBK470226/ (accessed Dec. 23, 2018).
(75) B. M. T. Pereira, M. P. Ogilvie, J. C. Gomez-Rodriguez, M. L. Ryan, D. Perla, A. C. Marttos, L. R. Pizano, M. G. McKenney, A review of ureteral injuries after external trauma, Scand. J. Trauma. Resusc. Emerg. Med. 18 (2010) 6.
(76) S. M. Goldman, C. M. Sandler, J. N. Corriere, E. J. McGuire, Blunt urethral trauma: a unified, anatomical mechanical classification, J. Urol. 157 (1997) 85-89.
(77) V. C. Onuora, A. H. Koko, A. S. Abdelwahab, Major injuries to the urinary tract in association with childbirth, East Afr. Med. J. 74 (1997) 523-526.
(78) W. S. Cobb, J. M. Burns, K. W. Kercher, B. D. Matthews, H. J. Norton, B. T. Heniford, Normal intraabdominal pressure in healthy adults, J. Surg. Res. 129 (2005) 231-235.
(79) B. L. De Keulenaer, J. J. De Waele, B. Powell, M. Malbrain, What is normal intra-abdominal pressure and how is it affected by positioning, body mass and positive end-expiratory pressure?, Intensive Care Med. 35 (2009) 969-976.
(80) S. P. Elliott, J. W. McAninch, Ureteral injuries: external and iatrogenic, Urol. Clin. 33 (2006) 55-66.

All publications mentioned herein (e.g. those above and U.S. Patent Publication No. 20160331564 etc.) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of making a hybrid hydrogel composition comprising:
combining together:
a crosslinkable biopolymer;
a crosslinkable synthetic or semi-synthetic polymer;
a crosslinking agent; and
crosslinking the biopolymer to the synthetic or semi-synthetic polymer so as to form a hybrid hydrogel,
so that the hybrid hydrogel composition is formed.

2. The method of claim 1, wherein amounts of the crosslinkable biopolymer and amounts of the crosslinkable synthetic or semi-synthetic polymer are selected so that the hybrid hydrogel comprises the biopolymer coupled to from 0.5-8% of the synthetic or semi-synthetic polymer.

3. The method of claim 2, wherein the biopolymer comprises at least one of:
an albumin, an alginate, a chitosan, a pectin, a cellulose, any other polysaccharide, a fibrin, a collagen and a gelatin, any other protein, said biopolymer having a first moiety that is crosslinkable to a second moiety on the synthetic or semi-synthetic polymer.

4. The method of claim 3, wherein the synthetic or semi-synthetic polymer comprises a polyethylene glycol or its derivatives, a polypropylene glycol or its derivatives or a cyanoacrylate or its derivatives.

5. The method of claim 4, wherein the hybrid hydrogel is crosslinked in vivo such that the hybrid hydrogel forms a solid cast that adheres to wet tissue interfaces contacting the hybrid hydrogel.

6. The method of claim 2, wherein:
amounts of the crosslinkable biopolymer and amounts of the crosslinkable synthetic or semi-synthetic polymer are selected so that the hybrid hydrogel composition exhibits an adhesion strength to tissues that is at least two fold greater than adhesion to tissues observed with the biopolymer not crosslinked to the synthetic polymer; and
amounts of the crosslinkable biopolymer and amounts of the crosslinkable synthetic polymer are selected so that the hybrid hydrogel composition exhibits a compression modulus that is at least two fold greater than the compression modulus of the biopolymer not crosslinked to the synthetic polymer.

7. The method of claim 2, wherein the hybrid hydrogel composition exhibits:
a tensile modulus of at least 150-350 kPa;
a compression modulus of at least 150-350 kPa; and/or
a storage modulus of at least 5-10 kPa.

8. The method of claim 1, wherein the method further comprises combining a bioactive agent such as a drug, a polypeptide, a polynucleotide or a cell with the crosslinkable biopolymer and the crosslinkable synthetic polymer.

9. The method of claim 1, wherein the crosslinking agent facilitates a photochemical crosslinking reaction.

10. A composition of matter comprising:
a crosslinkable biopolymer;
a crosslinkable synthetic polymer;
polymeric monomers; and
a crosslinking agent.

11. The composition of claim 11, wherein amounts of the crosslinkable biopolymer, the crosslinkable synthetic polymer, the polymeric monomers and the crosslinking agent in the composition are such that so that, upon crosslinking, a hybrid hydrogel polymer is formed that comprises the biopolymer covalently crosslinked to 0.5-8% of the synthetic polymer.

12. The composition of claim 11, wherein:
the biopolymer comprises at least one of an albumin, an alginate, a chitosan, a pectin, a cellulose, any other polysaccharide a fibrin, a collagen and a gelatin, any other protein, said biopolymer having a first moiety that is couplable to a second moiety on the synthetic polymer; and
the synthetic polymer comprises at least one of a polyethylene glycol or a polypropylene glycol.

13. The composition of claim 12, wherein upon crosslinking, a hybrid polymer hydrogel is formed that exhibits a compression modulus that is at least 2-fold greater than the compression modulus exhibited by a hydrogel formed from the biopolymer not crosslinked to the synthetic polymer.

14. The composition of claim 13, wherein upon crosslinking the composition forms a solid cast adhered to in vivo wet tissues contacting the hybrid hydrogel.

15. The composition of claim 12, wherein amounts of synthetic polymer disposed in the hybrid hydrogel are such that, upon crosslinking, a hybrid polymer hydrogel composition is formed that exhibits an adhesion strength to in vivo wet tissues that is at least two fold greater than adhesion strength to in vivo wet tissues observed with the biopolymer not crosslinked to the synthetic polymer.

16. A method of adhering a first tissue interface to a second tissue interface, the method comprising:
(a) forming a composition of matter comprising:
a crosslinkable biopolymer;
a crosslinkable synthetic polymer; and
a crosslinking agent;
(b) disposing the composition of (a) at a site where the composition is in contact with the first tissue interface and the second tissue interface; and
(c) crosslinking the composition of (a) at the site where the composition is in contact with the first tissue interface and the second tissue interface such that:
the crosslinked composition forms a hybrid polymer hydrogel consisting of the biopolymer covalently coupled to from 0.5-8% of the synthetic polymer; and
the crosslinked composition of adheres the first tissue interface to the second tissue interface.

17. The method of claim 16, wherein:
the biopolymer comprises gelatin methacrylate (GelMA) in amounts from 10% (w/v) to 30% (w/v); and
the crosslinkable synthetic polymer comprises a poly(ethylene glycol) diacrylate (PEGDA).

18. The method of claim 17, wherein the hybrid polymer hydrogel exhibits an adhesion strength between the first tissue interface and the second tissue interface of at least 50 kPa, at least 75 kPa or at least 100 kPa.

19. The method of claim 18, wherein the hybrid polymer hydrogel exhibits:
a tensile modulus of at least 150-350 kPa;
a compression modulus of at least 150-350 kPa; and/or
a storage modulus of at least 5-10 kPa.

20. The method of claim 19, wherein the composition further comprises a bioactive agent.
